(19) United States
(12) Patent Application Publication
(10) Pub. No.: US 2025/0152962 A1
(43) Pub. Date: May 15, 2025

(54) Oral Therapy Device

(71) Applicant: SHENZHEN KAIYAN MEDICAL EQUIPMENT CO., LTD, Shenzhen (CN)

(72) Inventors: ALAIN DIJKSTRA, Amstelveen (NL); YE QI, Shenzhen (CN)

(73) Assignee: SHENZHEN KAIYAN MEDICAL EQUIPMENT CO., LTD, Shenzhen (CN)

(21) Appl. No.: 18/602,116

(22) Filed: Mar. 12, 2024

(30) Foreign Application Priority Data
Nov. 10, 2023 (CN) 2023230395654

(51) Int. Cl. A61N 5/06; A61C 1/08; A61C 19/06

(52) U.S. Cl. CPC A61N 5/0603; A61C 1/081; A61C 19/06; A61N 5/0624; A61N 2005/0606; A61N 2005/0626; A61N 2005/0647; A61N 2005/0652; A61N 2005/0659; A61N 2005/0661; A61N 2005/0663

(57) ABSTRACT

Embodiments of the present invention provide an oral therapy device. The oral therapy device includes a therapeutic unit configured to be located inside an oral cavity of a user, the therapeutic unit including a plurality of stimulation elements provided thereupon, and a contact layer configured to make contact with inner surfaces of the oral cavity, the contact layer shaped as concaved inwards towards a housing container of the therapeutic unit. The oral therapy device further includes a control module electrically connected to the therapeutic unit through an electrical conductor. Also, the oral therapy device includes an electrical power source provided within the control module, the electrical power source configured to provide electrical power to the plurality of stimulation elements. Furthermore, the control module is configured to regulate the supply of the electrical power to the plurality of stimulation elements to modify operational characteristics of the plurality of stimulation elements.

### TECHNICAL FIELD

The present invention generally relates to a device that incorporates innovative features and functionalities to improve the effectiveness and convenience of oral hygiene routines and oral care therapies. More specifically, the present invention relates to a device designed for administering oral therapies to enhance oral hygiene practices and promote overall oral health by utilizing the application of various stimulation elements such as irradiation sources, heating elements, cooling elements, ultrasonic wave generators, vibratory elements, electrodes, and the like.

### BACKGROUND ART

Preserving ideal oral hygiene is essential in warding off a range of dental issues, such as cavities, gum diseases, soreness in the throat or mouth, and unpleasant breath. Furthermore, the oral cavity may suffer from other issues such as gum decay, toothaches, inflammation, allergic reactions, and the like. The oral care therapy devices designed for providing oral therapy to the mouth of a user, are readily available in the art. Furthermore, several compositions, such as toothpaste and chemical solutions, aimed at eliminating germs from the mouth, are also widely known. Additionally, pharmacies offer a range of drugs for treating mouth soreness. However, these products have notable drawbacks, including the risk of allergies from prolonged use of medications, unintended ingestion of the compositions, choking hazards with relatively small-sized devices, staining of teeth and erosion of tooth enamel, and development of drug resistance in the oral cavity. Furthermore, medical professionals employ diverse chemicals and equipment to treat oral ailments, but these procedures are both time-consuming and expensive.

U.S. Pat. No. 8,900,282B2 discloses a light therapy apparatus designed for delivering light to specific tissues in the dental and maxillofacial areas of a patient. The apparatus includes a support that aligns with anatomical features on the head of the patient, with one or more light sources mounted on the support. These light sources, embodied as arrays of LEDs, illuminate targeted tissues externally, avoiding the need for intra-oral placement. Further, the support comprises an intra-oral tray connected to an extra-oral bridge, where a light source (LED array) is affixed to the extra-oral bridge. Another configuration involves a headset registering on the nose and ears of the patient, with a mounted LED array for light delivery. The invention also encompasses methods for preparing the light therapy apparatus and delivering light therapy to the dental and maxillofacial areas. Overall, the innovation provides a non-intrusive and versatile approach to light therapy in the specified anatomical regions.

While US′282 offers light therapy for the oral cavity, the suggested solutions predominantly concentrate on the teeth area of the individual. However, the disclosed apparatuses cannot address soreness within the mouth. Furthermore, the light therapy is administered externally to the mouth, which may not be as effective as delivering the light therapy directly within the oral cavity.

Therefore, there is a need in the art for an oral therapy device that does not suffer from the aforementioned deficiencies.

### OBJECTS OF THE INVENTION

Some of the objects of the invention are as follows:

An object of the present invention is to provide a device that delivers therapy in the oral cavity of a user through several stimulation elements.

Another object of the present invention is to provide a device that can be fastened to a body portion of the user, while in use.

Another object of the present invention is to provide a device that is simple in construction, relatively inexpensive to purchase, and relatively convenient to use and carry.

Another object of the present invention is to provide a device that can be controlled using a user interface provided with the device.

It is also an object of the invention to provide a device that can also be controlled through a wired or a wireless connection using a user computing device such as a smartphone, a tablet, a notebook, and the like.

### SUMMARY OF THE INVENTION

According to a first aspect of the present invention, there is provided an oral therapy device. The oral therapy device includes a therapeutic unit configured to be located inside an oral cavity of a user, the therapeutic unit including a plurality of stimulation elements provided thereupon, and a contact layer configured to make contact with inner surfaces of the oral cavity, the contact layer shaped as concaved inwards towards a housing container of the therapeutic unit. The oral therapy device further includes a control module electrically connected to the therapeutic unit through an electrical conductor. Also, the oral therapy device includes an electrical power source provided within the control module. The electrical power source is configured to provide electrical power to the plurality of stimulation elements. Furthermore, the control module is configured to regulate the supply of the electrical power to the plurality of stimulation elements to modify the operational characteristics of the plurality of stimulation elements.

In one embodiment of the invention, the oral therapy device further includes a fastening arrangement coupled to the control module, the fastening arrangement configured to fasten the control module to a portion of a body of the user.

In one embodiment of the invention, the fastening arrangement includes a hook portion configured to conform with a shape of an ear of the user.

In one embodiment of the invention, the oral therapy device further includes an outer conduit connecting the control module with the therapeutic unit, the outer conduit including therewithin the electrical conductor and an inner conduit, wherein the control module further includes a composition container configured to store therewithin a therapeutic composition, the inner conduit configured to transfer the therapeutic composition from the composition container and into the oral cavity of the user.

In one embodiment of the invention, the control module further includes a pump configured to pump the therapeutic composition from the composition container and into the inner conduit.

In one embodiment of the invention, the therapeutic composition is selected from a group consisting of water, brine solution, pharmaceutical compositions, gels, emulsions, and combinations thereof.

In one embodiment of the invention, the plurality of stimulation elements is selected from a group consisting of irradiation sources, heating elements, cooling elements, vibration elements, ultrasonic wave generators, electrodes, spray nozzles, brushes, and combinations thereof.

In one embodiment of the invention, the irradiation sources are configured to emit electromagnetic radiation in a wavelength range of 300 nm to 1200 nm.

In one embodiment of the invention, portions of the contact layer above the irradiation sources are diaphanous.

In one embodiment of the invention, the control module further includes a user interface configured to receive control input signals from the user, for modifying the operational characteristics of the plurality of stimulation elements.

In one embodiment of the invention, the control module further includes a communication interface configured to receive control input signals from a user computing device, over a communication network, for modifying the operational characteristics of the plurality of stimulation elements.

In one embodiment of the invention, the electrical power source comprises rechargeable batteries.

According to a second aspect of the present invention, there is provided an oral therapy device. The oral therapy device includes a therapeutic unit configured to be located inside an oral cavity of a user, the therapeutic unit including a plurality of stimulation elements provided thereupon. The oral therapy device further includes a control module electrically connected to the therapeutic unit through an electrical conductor. Furthermore, the oral therapy device includes a fastening arrangement coupled to the control module, the fastening arrangement configured to fasten the control module to a portion of a body of the user. The oral therapy device also includes an electrical power source provided within the control module, the electrical power source configured to provide electrical power to the plurality of stimulation elements. Furthermore, the control module is configured to regulate the supply of the electrical power to the plurality of stimulation elements to modify operational characteristics of the plurality of stimulation elements.

In one embodiment of the invention, the control module includes a composition container configured to store therewithin a therapeutic composition, the control module connected to the therapeutic unit through an outer conduit including therewithin an inner conduit and the electrical conductor.

In one embodiment of the invention, the fastening arrangement includes a hook portion configured to conform with a shape of an ear of the user.

In one embodiment of the invention, the control module further includes a user interface configured to receive control input signals from the user, for modifying the operational characteristics of the plurality of stimulation elements.

In one embodiment of the invention, the control module further includes a communication interface configured to receive control input signals from a user computing device, over a communication network, for modifying the operational characteristics of the plurality of stimulation elements.

According to a third aspect of the present invention, there is provided a method of using an oral therapy device. The method includes providing the oral therapy device. The oral therapy device includes a therapeutic unit configured to be located inside an oral cavity of a user, the therapeutic unit including a plurality of stimulation elements provided thereupon, and a contact layer configured to make contact with inner surfaces of the oral cavity, the contact layer shaped as concaved inwards towards a housing container of the therapeutic unit. The oral therapy device further includes a control module electrically connected to the therapeutic unit through an electrical conductor. Also, the oral therapy device includes an electrical power source provided within the control module, the electrical power source configured to provide electrical power to the plurality of stimulation elements. Furthermore, the control module is configured to regulate the supply of the electrical power to the plurality of stimulation elements to modify operational characteristics of the plurality of stimulation elements. The method further includes locating the therapeutic unit in the oral cavity of the user, wherein the contact layer adheres to a predetermined region of the inner surfaces of the oral cavity due to a vacuum created by the concaved inwards shape of the contact layer. The method also includes regulating, through the control module, the supply of the electrical power to the plurality of stimulation elements to modify the operational characteristics of the plurality of stimulation elements.

In one embodiment of the invention, the method further includes fastening the control module to a portion of a body of the user through a fastening arrangement coupled to the control module.

In one embodiment of the invention, the step of regulating the power supply further includes receiving, by the control module, control input signals from the user, through one or more of a user interface provided with the control module and a communication interface over a communication network, for modifying the operational characteristics of the plurality of stimulation elements.

In the context of the specification, the term “processor” refers to one or more of a microprocessor, a microcontroller, a general-purpose processor, a Field Programmable Gate Array (FPGA), a Neural Processing Unit (NPU), a Graphics Processing Unit (GPU), a Tensor Processing Unit (TPU), an Application Specific Integrated Circuit (ASIC), and the like.

In the context of the specification, the phrase “memory unit” refers to volatile storage memory, such as Static Random Access Memory (SRAM) and Dynamic Random Access Memory (DRAM) of types such as Asynchronous DRAM, Synchronous DRAM, Double Data Rate SDRAM, Rambus DRAM, and Cache DRAM, etc.

In the context of the specification, the phrase “storage device” refers to a non-volatile storage memory such as EPROM, EEPROM, flash memory, or the like.

In the context of the specification, the phrase “communication interface” refers to a device or a module enabling direct connectivity via wires and connectors such as USB, HDMI, VGA, or wireless connectivity such as Bluetooth or Wi-Fi, or Local Area Network (LAN) or Wide Area Network (WAN) implemented through TCP/IP, IEEE 802.x, GSM, CDMA, LTE, or other equivalent protocols.

In the context of this specification, terms like “light”, “radiation”, “irradiation”, “emission” and “illumination”, etc. refer to electromagnetic radiation in frequency ranges varying between the Ultraviolet (UV) frequencies and Infrared (IR) frequencies and wavelengths (including all visible light frequencies and wavelengths), wherein the range is inclusive of UV and IR frequencies and wavelengths. It is to be noted here that UV radiation can be categorized in several manners depending on respective wavelength ranges, all of which are envisaged to be under the scope of this invention. For example, UV radiation can be categorized as, Hydrogen Lyman-α (122-121 nm), Far UV (200-122 nm), Middle UV (300-200 nm), and Near UV (400-300 nm). The UV radiation may also be categorized as UVA (400-315 nm), UVB (315-280 nm), and UVC (280-100 nm) Similarly, IR radiation may also be categorized into several categories according to respective wavelength ranges which are again envisaged to be within the scope of this invention. A commonly used subdivision scheme for IR radiation includes Near IR (0.75-1.4 μm), Short-Wavelength IR (1.4-3 μm), Mid-Wavelength IR (3-8 μm), Long-Wavelength IR (8-15 μm) and Far IR (15-1000 μm).

In the context of the specification, the term “polymer” or “plastic” refers to a material made up of long chains of organic molecules (having eight or more organic molecules) including, but not limited to, carbon, nitrogen, oxygen, and hydrogen as their constituent elements. The term polymer is envisaged to include both naturally occurring polymers such as wool, and synthetic polymers such as polyethylene and nylon.

In the context of the specification, the phrase “diaphanous material” refers to a material that allows at least a portion of one or more forms of electromagnetic radiation (such as Infrared, Ultraviolet, X-rays, Visible Light, Microwaves, Radio Waves, etc.) to pass through them. The diaphanous materials can be transparent (allowing one or more forms of electromagnetic radiation to pass through with minimal scattering) or translucent (allowing one or more forms of electromagnetic radiation to pass through with appreciable diffusion or scattering). Diaphanous materials can be dense, like glass, or have an open structure, like wire mesh or a woven fabric.

In the context of the specification, “Light Emitting Diodes (LEDs)” refer to semiconductor diodes capable of emitting electromagnetic radiation when supplied with an electric current. LEDs are characterized by their superior power efficiencies, smaller sizes, rapidity in switching, physical robustness, and longevity when compared with incandescent or fluorescent lamps. In that regard, one or more LEDs may be through-hole type LEDs (generally used to produce electromagnetic radiations of red, green, yellow, blue, and white colors), Surface Mount Technology (SMT) LEDs, Bi-color LEDs, Pulse Width Modulated RGB (Red-Green-Blue) LEDs, and high-power LEDs, etc.

Materials used in the LEDs may vary from one embodiment to another depending upon the frequency of radiation required. Different frequencies can be obtained from LEDs made from pure or doped semiconductor materials. Commonly used semiconductor materials include nitrides of Silicon, Gallium, Aluminum, Boron, Zinc Selenide, etc. in pure form or doped with elements such as Aluminum and Indium, etc. For example, red and amber colors are produced from Aluminum Indium Gallium Phosphide (AlGaInP) based compositions, while blue, green, and cyan use Indium Gallium Nitride based compositions. White light may be produced by mixing red, green, and blue lights in equal proportions, while varying proportions may be used to generate a wider color gamut. White and other colored lightings may also be produced using phosphor coatings such as Yttrium Aluminum Garnet (YAG) in combination with a blue LED to generate white light and Magnesium-doped potassium fluorosilicate in combination with a blue LED to generate red light. Additionally, near Ultraviolet (UV) LEDs may be combined with europium-based phosphors to generate red and blue lights and copper and zinc-doped zinc sulfide-based phosphors to generate green light.

In addition to conventional mineral-based LEDs, one or more LEDs may also be provided on an Organic LED (OLED) based flexible panel or an inorganic LED-based flexible panel. Such OLED panels may be generated by depositing organic semiconducting materials over Thin Film Transistor (TFT) based substrates. Further, a discussion on the generation of OLED panels can be found in Bardsley, J. N (2004), “International OLED Technology Roadmap”, IEEE Journal of Selected Topics in Quantum Electronics, Vol. 10, No. 1, that is included herein in its entirety, by reference. An exemplary description of flexible inorganic light-emitting diode strips can be found in granted U.S. Pat. No. 7,476,557 B2, titled “Roll-to-roll fabricated light sheet and encapsulated semiconductor circuit devices”, which is included herein in its entirety, by reference.

In several embodiments, the LEDs may also be micro-LEDs described through U.S. Pat. No. 8,809,126 B2, 8,846,457 B2, 8,852,467 B2, 8,415,879 B2, 8,877,101 B2, 9,018,833 B2 and their respective family members, assigned to NthDegree Technologies Worldwide Inc., which are included herein by reference, in their entirety. The LEDs, in that regard, may be provided as a printable composition of the micro-LEDs, printed on a substrate.

### BRIEF DESCRIPTION OF THE ACCOMPANYING DRAWINGS

The accompanying drawings illustrate the best mode for carrying out the invention as presently contemplated and set forth hereinafter. The present invention may be more clearly understood from a consideration of the following detailed description of the preferred embodiments taken in conjunction with the accompanying drawings wherein like reference letters and numerals indicate the corresponding parts in various figures in the accompanying drawings, and in which:

FIG. 1 illustrates a perspective view of an oral therapy device, in accordance with an embodiment of the present invention;

FIG. 2A illustrates an exploded view of the oral therapy device, in accordance with an embodiment of the present invention;

FIG. 2B illustrates an exploded view of the oral therapy device, in accordance with another embodiment of the present invention;

FIG. 3A illustrates an exploded view of a therapeutic unit of the oral therapy device, in accordance with an embodiment of the present invention;

FIG. 3B illustrates a partially exploded view of an outer conduit of the oral therapy device, in accordance with an embodiment of the present invention;

FIG. 3C illustrates an exploded view of a control module of the oral therapy device, in accordance with an embodiment of the present invention;

FIG. 4 illustrates a user using the oral therapy device, in accordance with an embodiment of the present invention;

FIG. 5 illustrates a user using the oral therapy device, in accordance with another embodiment of the present invention;

FIG. 6 illustrates a user using the oral therapy device, in accordance with another embodiment of the present invention;

FIG. 7 illustrates a user using the oral therapy device, in accordance with another embodiment of the present invention; and

FIG. 8 illustrates a method of using the oral therapy device, in accordance with an embodiment of the present invention.

### DETAILED DESCRIPTION

Embodiments of the present invention disclosure will be described more fully hereinafter with reference to the accompanying drawings in which like numerals represent like elements throughout the figures, and in which example embodiments are shown.

The detailed description and the accompanying drawings illustrate the specific exemplary embodiments by which the disclosure may be practiced. These embodiments are described in detail to enable those skilled in the art to practice the invention illustrated in the disclosure. It is to be understood that other embodiments may be utilized, and other changes may be made, without departing from the spirit or scope of the present disclosure. The following detailed description is therefore not to be taken in a limiting sense, and the scope of the present invention disclosure is defined by the appended claims. Embodiments of the claims may, however, be embodied in many different forms and should not be construed as limited to the embodiments set forth herein.

Embodiments of the present invention provide an oral therapy device. The oral therapy device includes a therapeutic unit that is configured to be located within the oral cavity of the user and provide therapy within the oral cavity. Furthermore, the therapy may be provided by several stimulation elements provided with the therapeutic unit. The stimulation elements may include irradiation sources, heating elements, cooling elements, vibratory elements, spray nozzles, brushes, ultrasonic wave generators, electrodes, and the like. A contact layer may also be provided upon the stimulation elements, where the contact layer would make contact with the inner surfaces of the oral cavity. The contact layer may be shaped concaved inwards to enable the contact layer to adhere to a specific region of the inner surfaces due to the creation of a vacuum by the concaved inwards shape. In the case of the stimulation elements including irradiation sources, portions of the contact layer above the irradiation sources may be made diaphanous. In the case of the stimulation elements including spray nozzles and brushes, portions of the contact layer above the spray nozzles and the brushes may be provided with apertures for the fluid sprayed by the nozzles or the brushes to make contact with the inner surfaces.

The oral therapy device further includes a control module that may be provided with an electrical power source for providing electrical power to the plurality of stimulation elements, a composition container for storing a therapeutic composition, a user interface for receiving control input signals, a processor, a memory unit, and a communication interface for implementing a control architecture. Furthermore, the control module is connected to the therapeutic unit through an outer conduit. The outer conduit in that regard at least encloses therewithin an electrical conductor connecting the electrical power source to the stimulation elements. In several embodiments, the outer conduit may also enclose an inner conduit configured to transfer the therapeutic composition from the composition container and into the oral cavity. The therapeutic composition may be delivered through a dispensing hole in the inner conduit or the therapeutic unit. Alternatively, the therapeutic composition may be delivered through the spray nozzles and/or brushes provided as stimulation elements. The control module may also be coupled with a fastening arrangement, such as a hook portion, which may conform with the shape of an ear of a user so that the oral therapy device may be fastened to the ear during the use of the oral therapy device. The fastening arrangement may also be embodied as a deformable band that may be fastened to the head or the neck of the user.

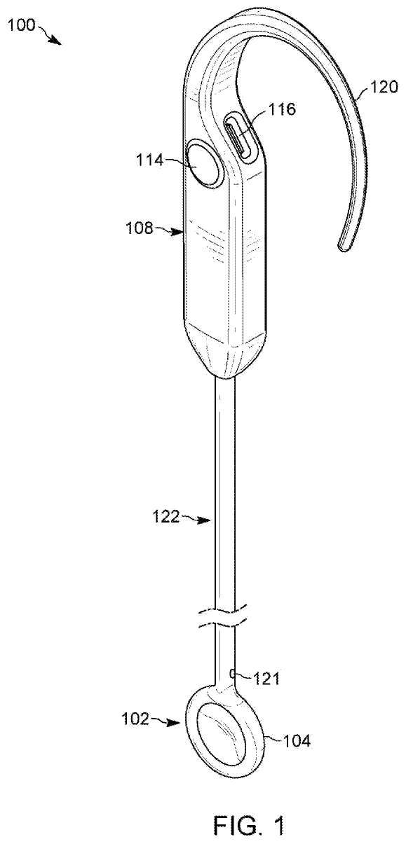

Several embodiments of the present invention will now be elucidated in detail with the help of FIGS. 1-8 as references.

FIG. 1 illustrates a perspective view of an oral therapy device 100 (hereinafter also referred to as “the device 100”), in accordance with an embodiment of the present invention. The device 100 includes a therapeutic unit 102. The therapeutic unit 102 is configured to be located within the oral cavity of a user for providing therapy within the oral cavity of the user. In that regard, the therapy may be contact-based or non-contact-based and provided to the inner surfaces of the oral cavity. Some of the examples of the inner surfaces include teeth, tongue, gums, inner surfaces of cheeks, jaw structure, upper throat region, and the like. The therapeutic unit 102 includes a housing container 104 configured to enclose several elements of the therapeutic unit 102 which will be discussed in the following discussion.

The device 100 further includes a control module 108. The control module 108 is connected to the therapeutic unit 102 through an outer conduit 122. The outer conduit 122 is envisaged to be made up of a pliable material so that the outer conduit 122 can be twisted and turned as per requirements during usage of the device 100. In that regard, the outer conduit 122 may be made up of a thin sheet of metal or a pliable polymeric material such as Polyvinyl chloride (PVC), Polyethylene (PE), Polypropylene (PP), Polyurethane (PU), Silicone, etc. The outer conduit 122 is also shown to have a dispensing hole 121 that is configured to dispense a therapeutic composition into the oral cavity of the user.

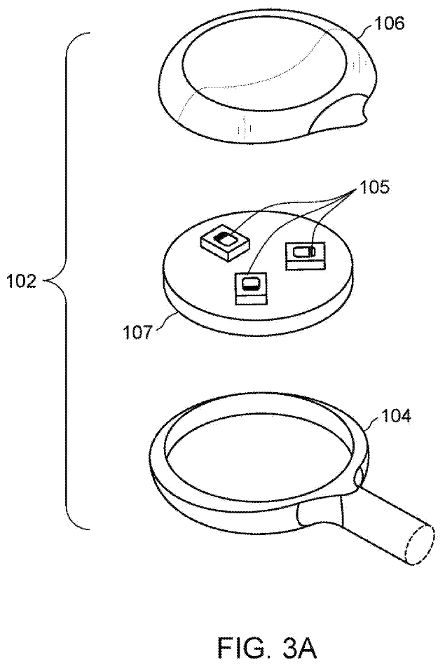

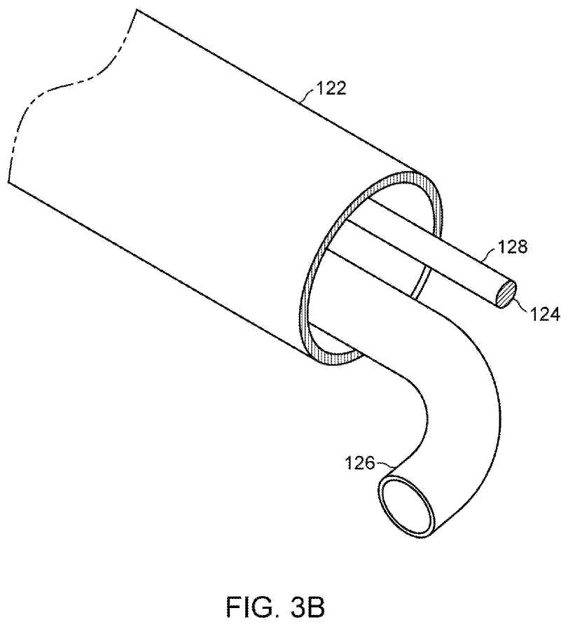

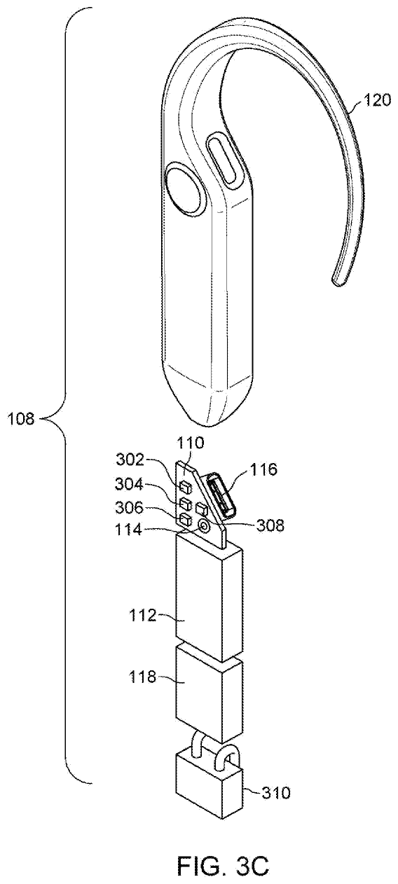

The control module 108 further includes a user interface 114. The user interface 114 may be embodied as a touch interface (resistive or capacitive type), a single control button, or a group of control toggles. The control module 108 is further depicted to include a data and power transfer port 116. The data and power transfer port 116 in that regard, may be selected from a group consisting of USB-A, B, C type ports, or any other compatible charging or data transfer port. The data and power transfer port 116 may be configured to charge an electrical power source (See FIG. 3C) of the control module 108 or transfer data to and from a memory unit or a storage device (See FIG. 3C) of the control module 108.

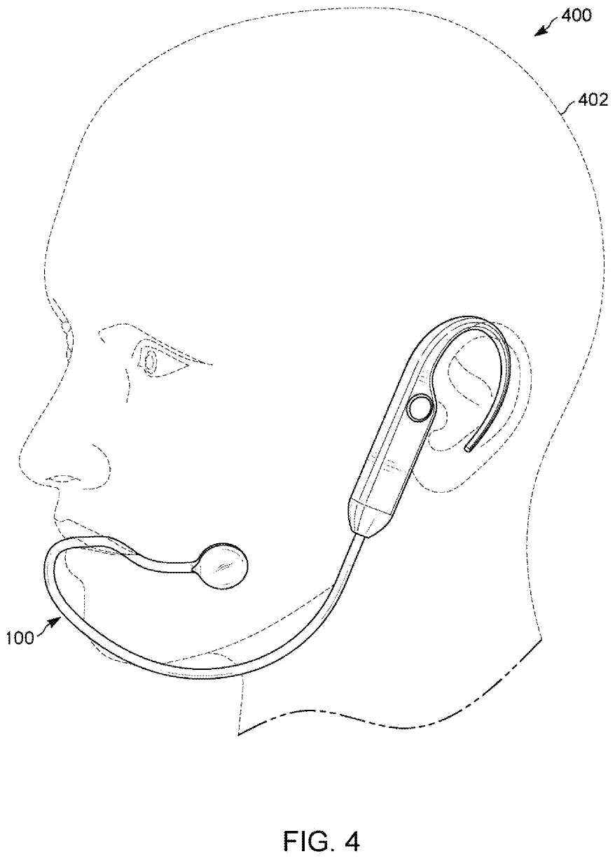

Furthermore, a fastening arrangement 120 is coupled to the control module 108. The fastening arrangement 120 is configured to fasten the control module 108 to a portion of the body of the user (See FIGS. 4, 5, 6, and 7). In the illustrated embodiment of the device 100, the fastening arrangement 120 includes a hook portion configured to conform with the shape of an ear of the user. However, the fastening arrangement 120 may be shaped in several different forms, for example, as a deformable band that can conform around a neck region or a head region of the user.

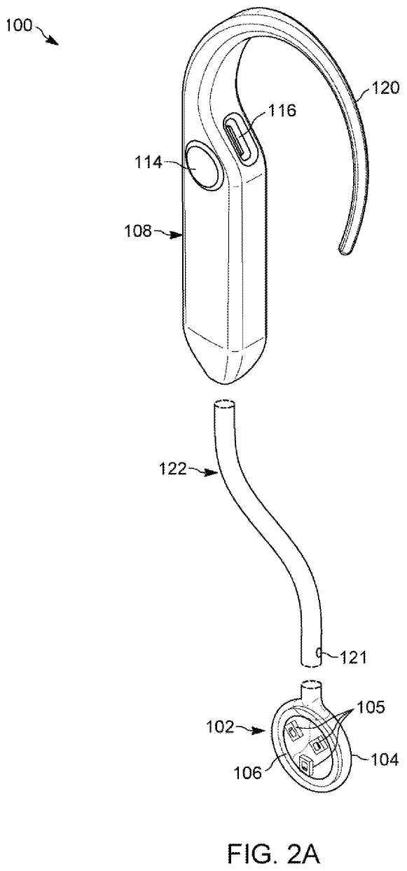

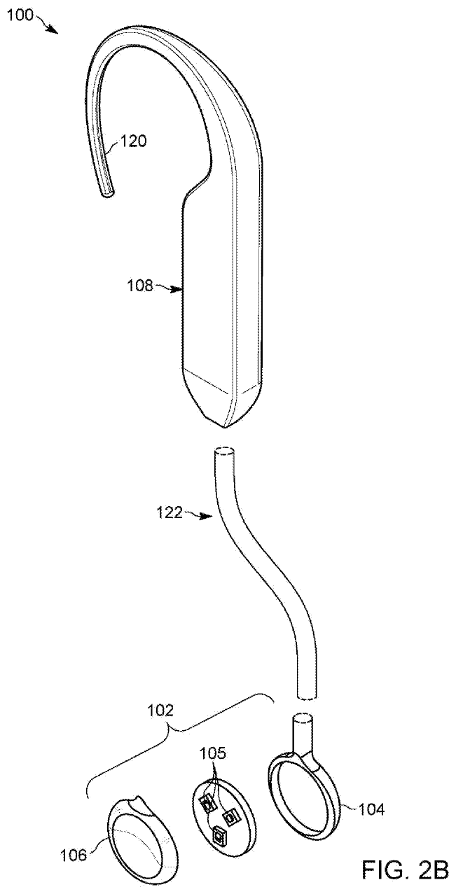

FIG. 2A illustrates an exploded view of the oral therapy device 100, in accordance with an embodiment of the present invention. FIG. 2B illustrates an exploded view of the oral therapy device 100, in accordance with another embodiment of the present invention. The housing container 104 encloses therewithin a plurality of stimulation elements 105. In several embodiments of the invention, the plurality of stimulation elements 105 may be selected from a group consisting of irradiation sources, heating elements, cooling elements, vibration elements, ultrasonic wave generators, electrodes, spray nozzles, brushes, and combinations thereof. Furthermore, irradiation sources may be selected from a group consisting of Light Emitting Diodes (LEDs), and lasers. Additionally, the irradiation sources may be configured to operate in one or more of a pulse mode and a continuous mode. In the case of a stimulation element being an irradiation source, the stimulation element would be emitting electromagnetic radiation.

In the case of a stimulation element being a vibration element, the stimulation element would be a vibrating head connected to an eccentric mass rotating motor or a linear resonant motor. In case of a stimulation element being a heating element, the stimulation element may be selected from a group consisting of metal heating elements, ceramic heating elements, semiconductor heating elements, thick film heating elements, polymer-based heating elements, composite heating elements, and combination heating elements. In the case of a stimulation element being a cooling element, the stimulation element may be a thermoelectric cooler, also known as a Peltier heat pump. In the case of a stimulation element being an ultrasonic wave generator, a wave generation head may consist of a quartz crystal fused with a metal plate. The quartz crystal may produce ultrasonic waves due to the piezoelectric effect. Ultrasonic wave therapy may be used in applications such as treatment of chronic pain, improvement in blood circulation, and tissue repair.

In case of a stimulation element being a spray nozzle, the spray nozzle may be configured to spray a therapeutic composition into the oral cavity. The therapeutic composition may include pure water or brine to perform oral hygiene operations such as scaling, flossing, mouthwash, etc. The therapeutic composition may also be an antiseptic or antibacterial mouthwash, a mouth ulcer gel, an anti-inflammatory composition containing salicylic acid or Non-Steroidal Anti-Inflammatory Drugs (NSAIDs) such as Ibuprofen, a tooth whitening and polishing composition, etc. The teeth whitening and polishing may be aided by brushes with rotating or vibrating bristles provided as one or more of the plurality of stimulation elements 105.

In the case of a stimulation element being an electrode, the stimulation element may be embodied as an open-ended conductor. The electrode may then be able to provide Transcutaneous Electrical Nerve Stimulation (TENS), Electronic Muscle Stimulation (EMS), and Microcurrent Electrical Therapy (MET) to the inner surfaces of the oral cavity. TENS therapy uses low-voltage currents to provide pain relief. Electrical impulses are delivered through electrodes placed on the surface of the body of the user. The electrodes are placed at or near nerves where the pain is located or at certain known trigger points. EMS therapy is similar to TENS therapy, the difference being that EMS is applied to key muscle groups instead of a generalized application. The electrical signals in EMS cause certain muscles to undergo contractions and tightening. Moreover, electrical impulses in EMS are stronger when compared with TENS therapy. MET in contrast uses a current of amplitude less than 1 milliampere and a frequency of 0.5 Hz and is indicated for the treatment of pain.

Furthermore, the therapeutic unit 102 includes a contact layer 106. The contact layer 106 is configured to make contact with the inner surfaces of the oral cavity of the user. In that regard, the contact layer 106 may be made up of a skin-friendly, food-grade material such as Polyurethane (PU), Silicone, Polylactic Acid (PLA), Polyglycolic Acid (PGA), etc. Furthermore, the contact layer 106 is shaped as concaved inwards towards the housing container 104. In that regard, the contact layer 106 is configured to adhere to a predetermined region of the inner surfaces of the oral cavity due to a vacuum created by the concaved inwards shape of the contact layer 106. Furthermore, it is envisaged that the contact layer 106 may be modified in conformance with the plurality of stimulation elements 105. For example, portions of the contact layer 106 above the irradiation sources may be made diaphanous. Furthermore, portions of the contact layer 106 above the electrodes may be made electrically conducting, above the heating and cooling elements may be made thermally conducting, above ultrasonic wave generators and vibratory elements may be made from material with high transmissibility. Moreover, apertures may be provided above the spray nozzles and the brushes.

FIG. 3A illustrates an exploded view of therapeutic unit 102 in accordance with an embodiment of the present invention. In addition to the housing container 104, and the contact layer 106, the therapeutic unit 102 also includes a Printed Circuit Board (PCB) 107 on which the plurality of stimulation elements 105 are electrically connected. FIG. 3B illustrates a partially exploded view of the outer conduit 122, in accordance with an embodiment of the present invention. The outer conduit 122 includes an inner conduit 126 and an electrical conductor 124. The electrical conductor 124 has been provided with an insulation layer 128 to prevent current leakage into the inner 126 or the outer 122 conduits. The inner conduit 126 is envisaged to be made up of a pliable material and the material of the inner conduit 126 may be selected from the materials listed for the outer conduit 122. The inner conduit 126 is configured to transfer the therapeutic composition from a composition container (See FIG. 3C) of the control module 108 and into the oral cavity of the user. The electrical conductor 124 electrically connects the control module 108 with the therapeutic unit 102 and consequently the plurality of stimulation elements 105 through the PCB 107.

FIG. 3C illustrates an exploded view of the control module 108, in accordance with an embodiment of the present invention. The control module 108 includes a second PCB 110 to which electrically connected are a processor 302, a memory unit 304 configured to store machine-readable instructions, a communication interface 306, a storage device 308, the user interface 114, and the data and power transfer port 116. The user interface 114 is configured to toggle the control module 108 between several operational states when pressure is applied to the user interface 114. The control module 108 further includes the electrical power source 112. The electrical power source 112 is configured to provide electrical power to the plurality of stimulation elements 105. In several embodiments of the invention, the electrical power source 112 may include rechargeable batteries such as Lithium-ion, Lithium-polymer, and Nickel-Metal-Hydride batteries. In several alternate embodiments of the invention, the electrical power source 112 may act as an adapter including rectifiers and step-down transformers to convert power from an AC power supply to DC power for the second PCB 110 and the plurality of stimulation elements 105.

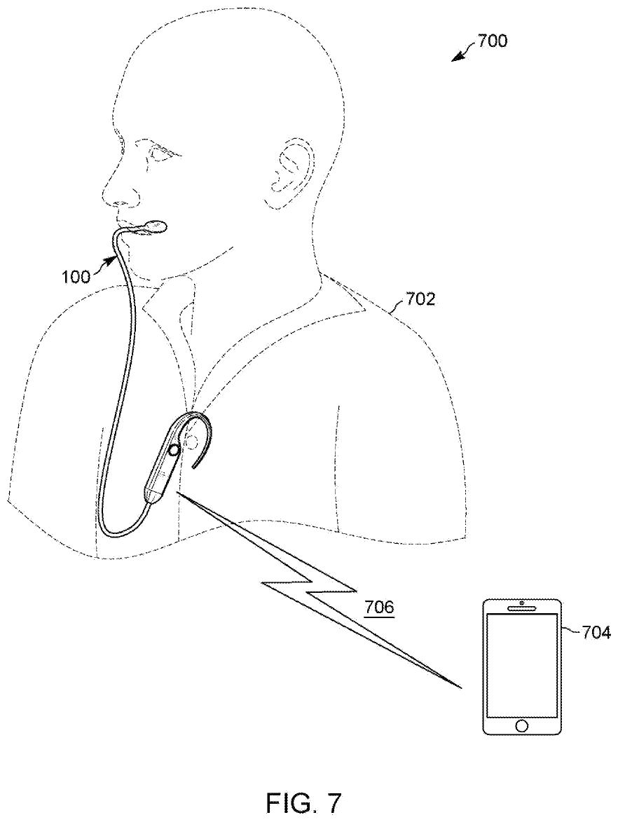

Furthermore, the processor 302 executing the machine-readable instructions may enable the control module 108 to regulate the supply of the electrical power to the plurality of stimulation elements 105 to modify the operational characteristics of the plurality of stimulation elements 105. Some of the non-limiting examples of the operational characteristics may include wavelength, intensity, period of pulses for the irradiation sources, temperature and time duration of activation of heating and cooling elements, frequency or acceleration of vibratory elements, frequency, and intensity of sound waves of the ultrasonic wave generators, applied voltage and current at the electrodes, etc. In that regard, the user interface 114 may be configured to receive control input signals from the user, for modifying the operational characteristics of the plurality of stimulation elements 105. Furthermore, the communication interface 306 may be configured to receive the control input signals from a user computing device, over a communication network (See FIG. 7), for modifying the operational characteristics of the plurality of stimulation elements 105.

The control module 108 further includes the composition container 118 configured to store therewithin the therapeutic composition. As discussed above, the therapeutic composition may be selected from a group consisting of water, brine solution, pharmaceutical compositions, gels, emulsions, and combinations thereof. The control module 108 further includes a pump 310 configured to pump the therapeutic composition from the composition container 118 and into the inner conduit 126. In several embodiments of the invention, the pump 310 may be selected from a group consisting of positive displacement pumps (such as peristaltic pumps, gear pumps, etc.) and centrifugal pumps.

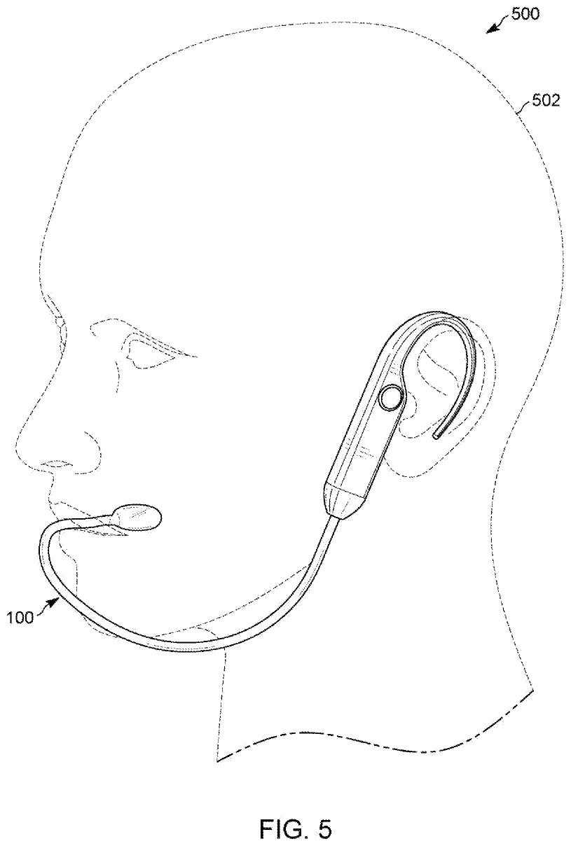

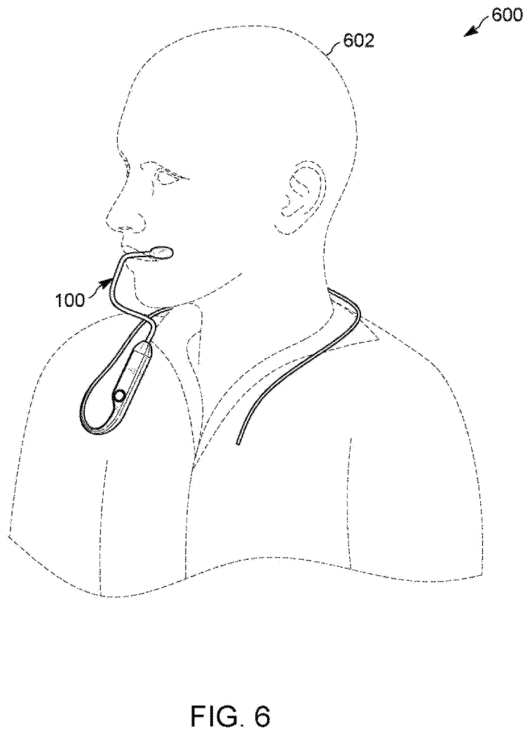

FIG. 4 illustrates a user 402 using the oral therapy device 100, in accordance with an embodiment 400 of the present invention. The therapeutic unit 102 has been located within the oral cavity and the hook portion of the fastening arrangement 120 has been fastened to the ear. The therapeutic unit 102 has been oriented toward the lower jaw of the user 402. FIG. 5 illustrates a user 502 using the oral therapy device 102, in accordance with another embodiment 500 of the present invention. The therapeutic unit 102 has been oriented toward the lower jaw of the user 502. FIG. 6 illustrates a user 602 using the oral therapy device 100, in accordance with another embodiment of the present invention. The fastening arrangement 120 includes a deformable band wrapped around the neck region of the user 602. FIG. 7 illustrates a user 702 using the oral therapy device 100, in accordance with another embodiment 700 of the present invention. The fastening arrangement 120 has been fastened to a shirt of the user 702. Furthermore, the control module 108 is in communication with a user computing device 704 through the communication interface 306 and a communication network 706. The user computing device 704 may be selected from a group consisting of a smartphone, a tablet, a notebook PC, a desktop PC, and the like.

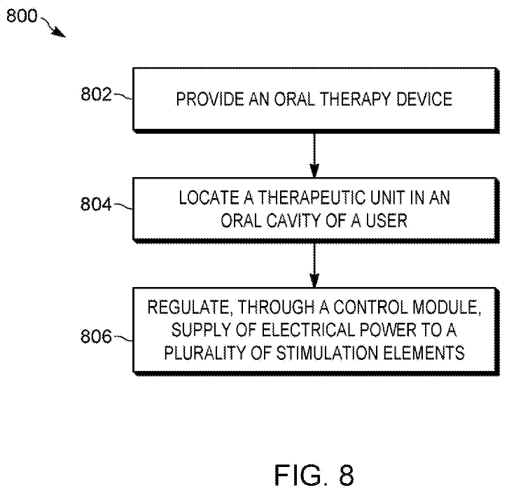

FIG. 8 illustrates a method 800 of using the oral therapy device, in accordance with an embodiment of the present invention. The method 800 begins at Step 802 when the device 100 as disclosed in the aforementioned embodiments is provided to a user. At Step 804, the therapeutic unit 102 is located within the oral cavity of the user. In several embodiments of the invention, the control module 108 is fastened to a portion of the body of the user through the fastening arrangement 120 coupled to the control module 108. Furthermore, the contact layer 106 adheres to a predetermined region of the inner surfaces of the oral cavity due to a vacuum created by the concaved inwards shape of the contact layer 106. In Step 806, the supply of the electrical power to the plurality of stimulation elements 105 is regulated through the control module 108 to modify the operational characteristics of the plurality of stimulation elements 105. In several embodiments of the embodiments, the control module 108 receives the control input signals from the user, through one or more of the user interface 114 and the communication interface 306 over the communication network 706, for modifying the operational characteristics of the plurality of stimulation elements 105.

The embodiments of the present invention as described above offer several advantages. For instance, the oral therapy device is simple in construction and convenient to use and transport. The same device can be used for providing a combination of therapies such as sterilization, heating, cooling, brushing, flossing, scaling, application of a pharmaceutical substance, etc for comprehensive oral care. The fastening arrangement allows the oral therapy device to be worn on the body leaving the hands free for other purposes. The oral therapy device can be operated in several operating modes where the modes may be toggled between using the user interface or a mobile application.

Various modifications to these embodiments are apparent to those skilled in the art, from the description and the accompanying drawings. The principles associated with the various embodiments described herein may be applied to other embodiments. Therefore, the description is not intended to be limited to the embodiments shown along with the accompanying drawings but is to provide the broadest scope consistent with the principles and the novel and inventive features disclosed or suggested herein. Accordingly, the invention is anticipated to hold on to all other such alternatives, modifications, and variations that fall within the scope of the present invention and appended claims.

1. An oral therapy device, comprising:
a therapeutic unit configured to be located inside an oral cavity of a user, the therapeutic unit comprising a plurality of stimulation elements provided thereupon, and a contact layer configured to make contact with inner surfaces of the oral cavity, the contact layer shaped as concaved inwards towards a housing container of the therapeutic unit;
a control module electrically connected to the therapeutic unit through an electrical conductor; and
an electrical power source provided within the control module, the electrical power source configured to provide electrical power to the plurality of stimulation elements,
wherein the control module is configured to regulate the supply of the electrical power to the plurality of stimulation elements to modify operational characteristics of the plurality of stimulation elements.

2. The oral therapy device as claimed in claim 1, further comprising a fastening arrangement coupled to the control module, the fastening arrangement configured to fasten the control module to a portion of a body of the user.

3. The oral therapy device as claimed in claim 2, wherein the fastening arrangement comprises a hook portion configured to conform with a shape of an ear of the user.

4. The oral therapy device as claimed in claim 1, further comprises an outer conduit connecting the control module with the therapeutic unit, the outer conduit comprising therewithin the electrical conductor and an inner conduit, wherein the control module further comprises a composition container configured to store therewithin a therapeutic composition, the inner conduit configured to transfer the therapeutic composition from the composition container and into the oral cavity of the user.

5. The oral therapy device as claimed in claim 4, wherein the control module further comprises a pump configured to pump the therapeutic composition from the composition container and into the inner conduit.

6. The oral therapy device as claimed in claim 4, wherein the therapeutic composition is selected from a group consisting of water, brine solution, pharmaceutical compositions, gels, emulsions, and combinations thereof.

7. The oral therapy device as claimed in claim 1, wherein the plurality of stimulation elements is selected from a group consisting of irradiation sources, heating elements, cooling elements, vibration elements, ultrasonic wave generators, electrodes, spray nozzles, brushes, and combinations thereof.

8. The oral therapy device as claimed in claim 7, wherein the irradiation sources are configured to emit electromagnetic radiation in a wavelength range of 300 nm to 1200 nm.

9. The oral therapy device as claimed in claim 7, wherein portions of the contact layer above the irradiation sources are diaphanous.

10. The oral therapy device as claimed in claim 1, wherein the control module further comprises a user interface configured to receive control input signals from the user, for modifying the operational characteristics of the plurality of stimulation elements.

11. The oral therapy device as claimed in claim 1, wherein the control module further comprises a communication interface configured to receive control input signals from a user computing device, over a communication network, for modifying the operational characteristics of the plurality of stimulation elements.

12. The oral therapy device as claimed in claim 1, wherein the electrical power source comprises rechargeable batteries.

13. An oral therapy device, comprising:
a therapeutic unit configured to be located inside an oral cavity of a user, the therapeutic unit comprising a plurality of stimulation elements provided thereupon;
a control module electrically connected to the therapeutic unit through an electrical conductor;
a fastening arrangement coupled to the control module, the fastening arrangement configured to fasten the control module to a portion of a body of the user; and
an electrical power source provided within the control module, the electrical power source configured to provide electrical power to the plurality of stimulation elements,
wherein the control module is configured to regulate the supply of the electrical power to the plurality of stimulation elements to modify operational characteristics of the plurality of stimulation elements.

14. The oral therapy device as claimed in claim 13, wherein the control module comprises a composition container configured to store therewithin a therapeutic composition, the control module connected to the therapeutic unit through an outer conduit comprising therewithin an inner conduit and the electrical conductor.

15. The oral therapy device as claimed in claim 13, wherein the fastening arrangement comprises a hook portion configured to conform with a shape of an ear of the user.

16. The oral therapy device as claimed in claim 13, wherein the control module further comprises a user interface configured to receive control input signals from the user, for modifying the operational characteristics of the plurality of stimulation elements.

17. The oral therapy device as claimed in claim 13, wherein the control module further comprises a communication interface configured to receive control input signals from a user computing device, over a communication network, for modifying the operational characteristics of the plurality of stimulation elements.

18. A method of using an oral therapy device, the method comprising:
providing the oral therapy device, the oral therapy device comprising:
a therapeutic unit configured to be located inside an oral cavity of a user, the therapeutic unit comprising a plurality of stimulation elements provided thereupon, and a contact layer configured to make contact with inner surfaces of the oral cavity, the contact layer shaped as concaved inwards towards a housing container of the therapeutic unit,
a control module connected to the therapeutic unit through an outer conduit, the outer conduit comprising therewithin an inner conduit and an electrical conductor, and
an electrical power source provided within the control module, the electrical power source configured to provide electrical power to the plurality of stimulation elements,
wherein the control module is configured to regulate the supply of the electrical power to the plurality of stimulation elements to modify operational characteristics of the plurality of stimulation elements;
locating the therapeutic unit in the oral cavity of the user, wherein the contact layer adheres to a predetermined region of the inner surfaces of the oral cavity due to a vacuum created by the concaved inwards shape of the contact layer; and
regulating, through the control module, the supply of the electrical power to the plurality of stimulation elements to modify the operational characteristics of the plurality of stimulation elements.

19. The method as claimed in claim 18, further comprising fastening the control module to a portion of a body of the user through a fastening arrangement coupled to the control module.

20. The method as claimed in claim 18, wherein the step of regulating the power supply further comprises receiving, by the control module, control input signals from the user, through one or more of a user interface provided with the control module and a communication interface over a communication network, for modifying the operational characteristics of the plurality of stimulation elements.
